(19) United States
(12) Patent Application Publication
(10) Pub. No.: US 2026/0365989 A1
(43) Pub. Date: Nov. 19, 2026

(54) EFFICIENT AND SCALABLE SUSPENSION IPSC CULTURE SYSTEM COMPLIANT WITH GOOD MANUFACTURING PRACTICES

(71) Applicant: Cedars-Sinai Medical Center, Los Angeles, CA (US)

(72) Inventors: Dhruv Sareen, Porter Ranch, CA (US); Hojae Lee, Los Angeles, CA (US); Raquel Martin-Ibanez, Los Angeles, CA (US)

(73) Assignee: Cedars-Sinai Medical Center, Los Angeles, CA (US)

(21) Appl. No.: 19/133,647

(22) PCT Filed: Dec. 8, 2023
(86) PCT No.: PCT/US2023/083092

§ 371 Date: May 28, 2025

Related U.S. Application Data
(60) Provisional application No. 63/431,153, filed on Dec. 8, 2022.

(51) Int. Cl. C12N 5/074; C12N 5/00

(52) U.S. Cl. CPC C12N 5/0696; C12N 5/0018; C12N 2509/10; C12N 2513/00; C12N 2531/00

(57) ABSTRACT

Described herein are cGMP methods of iPSC adaptation and culturing in suspension culture. Also described are iPSC expansion methods in suspension culture. Various embodiments provide for a method of adapting adherent induced pluripotent stem cells (iPSCs) to suspension iPSCs, comprising: (i) seeding about 75,000-800,000 iPSCs per ml cGMP feeder-free iPSC maintenance medium; (ii) culturing the iPSCs for about 2.5-3.S days to allow the formation of iPSC spheres; (iii) culturing the iPSC spheres for about 3.5-4.S days, feeding the iPSC spheres about daily with the cGMP feeder-free iPSC maintenance medium; (iv) passaging the iPSC spheres; (v) culturing about 80,000-120,000 iPSC spheres for about 3.5-4.5 days in the cGMP feeder free iPSC maintenance medium; (vi) culturing the iPSC sphere for about 2.5-3.S days, feeding the culture about daily with the cGMP feeder-free iPSC maintenance medium; (vii) repeating steps (iv)-(vi) at. least once.

### CROSS-REFERENCE TO RELATED APPLICATIONS

This application includes a claim of priority under 35 U.S.C. § 119(e) to U.S. provisional patent application No. 63/431,153, filed Dec. 8, 2022, the entirety of which is hereby incorporated by reference.

### STATEMENT REGARDING FEDERALLY SPONSORED RESEARCH OR DEVELOPMENT

This invention was made with Government support under Grant No. W81XWH-20-9-0022 awarded by the Department of Defense. The Government has certain rights in the invention.

### FIELD OF INVENTION

This invention relates to suspension culture system for iPSC manufacturing.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

For iPSC-derived cell therapy manufacturing, high-cost of scalable cell production and lack of analytic quality-control systems are one of bottlenecks. Traditional iPSC culture methods involve culturing them in two dimensional (2D) adherent culture systems. While these systems have been valuable in traditional research applications, (2D) iPSC culture systems are not amenable when moving towards commercial level scale-up for delivery to cellular therapy products. Further, line to line variability, long process and absence of optimal standards for scaling up are some challenges to overcome. While iPSC-derived cell therapy companies have come a long way over the past 5 years, there remains a big gap to scale up of iPSCs to create the final cell therapy product. This is an area that needs to be tackled. As such, there remains a need in the art for scalable iPSC manufacturing at commercial scale.

### SUMMARY OF THE INVENTION

The following embodiments and aspects thereof are described and illustrated in conjunction with compositions and methods which are meant to be exemplary and illustrative, not limiting in scope.

Various embodiments provide for a method of adapting adherent induced pluripotent stem cells (iPSCs) to suspension iPSCs, comprising:
(i) seeding about 75,000-800,000 iPSCs per ml cGMP feeder-free iPSC maintenance medium;
(ii) culturing the iPSCs for about 2.5-3.5 days to allow the formation of iPSC spheres;
(iii) culturing the iPSC spheres for about 3.5-4.5 days, feeding the iPSC spheres about daily with the cGMP feeder-free iPSC maintenance medium;
(iv) passaging the iPSC spheres;
(v) culturing about 80,000-120,000 iPSC spheres for about 3.5-4.5 days in the cGMP feeder-free iPSC maintenance medium;
(vi) culturing the iPSC sphere for about 2.5-3.5 days, feeding the culture about daily with the cGMP feeder-free iPSC maintenance medium;
(vii) repeating steps (iv)-(vi) at least once.

In various embodiments, passaging the iPSC spheres can comprise: collecting the iPSC spheres in tube (e.g., a conical tube); settling down iPSC spheres and removing extra medium; adding about 4-6 ml dissociation solution, wherein the dissociation solution comprises cell detachment solution of proteolytic and collagenolytic enzymes and does not contain mammalian or bacterial derived products, and incubating in an about 36-37 C degree water bath for about 3-12 min; dissociating iPSC spheres; straining the iPSC spheres through an about 35-45 um cell strainer and collecting the strained iPSC spheres; adding a quantity of cGMP feeder-free iPSC maintenance medium and a quantity of dissociation solution to the strained iPSC spheres; spinning a conical tube comprising the strained iPSC spheres, cGMP feeder-free iPSC maintenance medium and dissociation solution at about 250-350×g for about 4-6 min; removing supernatant and resuspending the iPSC spheres with fresh cGMP feeder-free iPSC maintenance medium.

In various embodiments, the method can comprise:
(i) seeding about 100,000-700,000 iPSCs per ml cGMP feeder-free iPSC maintenance medium;
(ii) culturing the iPSCs for about three days to allow the formation of iPSC spheres;
(iii) culturing the iPSC spheres for about four days, feeding the iPSC spheres daily with the cGMP feeder-free iPSC maintenance medium;
(iv) passaging the iPSC spheres;
(v) culturing about 100,000 iPSC spheres for about four days in the cGMP feeder-free iPSC maintenance medium;
(vi) culturing the iPSC sphere for about three days, feeding the culture daily with the cGMP feeder-free iPSC maintenance medium; and
(vii) repeating steps (iv)-(vi) at least once.

In various embodiments, passaging the iPSC spheres can comprise: collecting the iPSC spheres in tube (e.g., a conical tube); settling down iPSC spheres and removing extra medium; adding about 5 ml dissociation solution, wherein the dissociation solution comprises cell detachment solution of proteolytic and collagenolytic enzymes and does not contain mammalian or bacterial derived products, and incubating in a 37 degree water bath for about 5-10 min; dissociating iPSC spheres; straining the iPSC spheres through an about 40 um cell strainer and collecting the strained iPSC spheres; adding approximately equal amounts of cGMP feeder-free iPSC maintenance medium and dissociation solution to the strained iPSC spheres; spinning a conical tube comprising the strained iPSC spheres, cGMP feeder-free iPSC maintenance medium and dissociation solution at about 300×g for about 5 min; removing supernatant and resuspending the iPSC spheres with fresh cGMP feeder-free iPSC maintenance medium.

In various embodiments, the method can further comprise counting the iPSC spheres.

In various embodiments, the method can further comprise banking the iPSC spheres or further passaging the iPSC spheres.

In various embodiments, the suspension culture can be in an about 30 ml bioreactor.

Various embodiments of the invention provide for a method of culturing induced pluripotent stem cells (iPSCs) in suspension medium, comprising
(i) seeding about 80,000-120,000 iPSCs spheres per ml in cGMP feeder-free iPSC maintenance medium for about 3.5-4.5 days, wherein the iPSCs have been adapted from adherent iPSCs to suspended iPSCs;
(ii) culturing the iPSC spheres for about 2.5-3.5 days, feeding the iPSC sphere culture daily with cGMP feeder-free iPSC maintenance medium; and
(iii) passaging the iPSC spheres.

In various embodiments, passaging the iPSC spheres can comprise: collecting the iPSC spheres in tube (e.g., a conical tube); settling down spheres and removing extra medium; adding about 4-6 ml dissociation solution and incubating in an about 36-38 C degree water bath for about 3-12 min; dissociating iPSC spheres; straining the iPSC spheres through an about 35-45 um cell strainer; adding a quantity of cGMP feeder-free iPSC maintenance medium and a quantity of dissociation solution; spinning the tub at about 250-350×g for about 4-6 min; removing supernatant and resuspend the iPSC spheres with fresh cGMP feeder-free iPSC maintenance medium; and counting the iPSC spheres.

In various embodiments, the method can comprise:
(i) seeding about 100,000 iPSCs spheres per ml in cGMP feeder-free iPSC maintenance medium for about four days, wherein the iPSCs have been adapted from adherent iPSCs to suspended iPSCs;
(ii) culturing the iPSC spheres for about three days, feeding the iPSC sphere culture daily with cGMP feeder-free iPSC maintenance medium; and
(iii) passaging the iPSC spheres.

In various embodiments, passaging the iPSC spheres can comprise: collecting the iPSC spheres in tube (e.g., a conical tube); settling down spheres and removing extra medium; adding about 5 ml dissociation solution and incubating in a 37 C degree water bath for about 5-10 min; dissociating iPSC spheres; straining the iPSC spheres through an about 40 um cell strainer; adding approximately equal amounts of cGMP feeder-free iPSC maintenance medium and dissociation solution; spinning the tub at about 300×g for about 5 min; removing supernatant and resuspend the iPSC spheres with fresh cGMP feeder-free iPSC maintenance medium; and counting the iPSC spheres.

In various embodiments, the method can further comprise repeating steps (i)-(iii) at least once. In various embodiments, the method can further comprise repeating steps (ii)-(iii) at least once.

In various embodiments, the method can further comprise banking the cells or further passaging the cells.

In various embodiments, dissociating the cells can comprise dissociating with force by pipetting.

In various embodiments, the suspension culture can be in an about 30 ml bioreactor.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 (panels A-E) depicts adaptation of adherent to suspension cell. (A) Table of different seeding density for each line (0003iCTR and 628iCTR) and result of each density condition. (B) Sphere images of 3 consecutive passages from 0003iCTR and 628iCTR with 300 k/ml density. (C) Sphere size of each passage (1-3) from 2 lines (at least n=10 (n means independent spheres), Passage 1: present in the table, feeding daily and Passage 2-3: 1e5 cells/ml, 20 ml, Feeding: #5). (D) Flow plot of unstained control and SiPSC (0003iCTR, 300 k/ml at Passage 3) staining by OCT4 and SSEA antibodies. (E) Metabolic values of Glucose, Lactate and Ammonium of #5 feeding method in the media from Passage 3 of 0003iCTR(300 k/ml) (Glucose: red (1 g/L), blue (1.6 g/L), Lactate: red (2.5 g/L), blue (1.953 g/L), NH4+: red (2 mmol/L), blue (1.6 mmol/L)). mean±SD. Scale bar: 550 um.

FIG. 2 (panels A-F) depicts an optimal feeding method and monitoring analysis for suspension culture (A) Sphere size of each condition (at least n=10 (n means independent spheres), 1e5 cells/ml, 20 ml). (B) Cell expansion after normalization by #1 condition for fold change (n=9 (n means independent batches including 3 independent biological and technical replicates), 1e5 cells/ml, 20 ml, (Fold change values are presented in the graph)) (n.s.: not significant, *p<0.05, #p<0.05). (C) Flow plot of unstained control and SiPSC staining by OCT4 and SSEA antibodies. (D-F) Metabolic values of Glucose, Lactate and Ammonium (NH4+) of #1, 3 and 5 feeding methods in the media from SiPSC culture spinner flask (Glucose: red (1 g/L), blue (1.6 g/L), Lactate: red (2.5 g/L), blue (1.953 g/L), NH4+: red (2 mmol/L), blue (1.6 mmol/L); at least n=3 (n means independent batches including 3 independent biological and technical replicates) of each iPSC line). mean±SD. iPSC lines: 2AE8iCTR, 0003iCTR and 628iCTR.

FIG. 3 (panels A-B) depicts representative images of suspension cultures. (A) Representative sphere images of 0003iCTR, 2AE 8iCTR and 628iCTR. Scale bar: 550 um. (B) example of a process timeline.

FIG. 4 (panels A-C) depicts Standardization of metabolic media condition by NOVA analysis (A-C) Adherent cell culture based metabolic values of Glucose, Lactate and Ammonium (at least n=6, n means independent lines, limit values are presented in each graph). Error bars, mean±SD

FIG. 5 (panels A-B) depicts critical metabolomic candidate for sphere culture media monitoring. (A) Representative sphere images in negative condition (non-feeding) from culture day3 to day7. (B) Metabolic parameters of NOVA analysis in negative feeding methods (non-feeding) (at least n=3 (n means independent technical replicates); red dot means limit values of each parameter, blue dot means average values of 2D culture system (Glucose: red (1 g/L), blue (1.6 g/L), Lactate: red (2.5 g/L), blue (1.953 g/L), NH4+: red (2 mmol/L), blue (1.6 mmol/L)). mean±SD.

FIG. 6 (panels A-E) depicts monitoring of recovery with post-thawed cells (A) Table for cell viability for 2 weeks (2AE8iCTR). (B) Cell expansion rate between #1 and #5 feeding methods for 2 weeks (at least n=3 (n means independent biological repeats), 1e5 cells/ml, 20 ml volume). (C) Monitoring of sphere size for 2 weeks (at least n=10 (n means independent spheres), 1e5 cells/ml, 20 ml) (n.s.: not significant, **p<0.01, ****p<0.0001). (D) Flow plot of unstained control and SiPSC staining by OCT4 and SSEA antibodies at second week. mean±SD.

FIG. 7 depicts a summary of the sphere culturing and cell adaptation process timeline.

FIG. 8 shows representative karyotyping results. The genetic integrity and normal karyotype data with the suspension iPSCs shows that suspension iPSC banks maintain normal genetic integrity.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

An example of dissociation solution used herein is Accutase®, which is a cell detachment solution of proteolytic and collagenolytic enzymes and does not contain mammalian or bacterial derived products

An example of cGMP feeder-free iPSC maintenance medium is mTeSR Plus, which is a cGMP, feeder-free maintenance medium for human ES and iPS cells.

As example of a freezing reagent is CS10 from StemCell™, which is a serum-free, animal component-free and defined cryopreservation medium containing 10% dimethyl sulfoxide (DMSO). CS10 is designed to preserve cells in low temperature environments (−80 °C to −196 °C).

Described herein the invention provides for scalable iPSC manufacturing at commercial scale in 3D suspension culture platforms. Notably, iPSCs are very sensitive to shear stress magnitudes and we have discovered critical process parameters that allow for such a successful scale-up of iPSCs into suspension culture platform systems in for example, stirred-impeller mixed bioreactors.

By scaling up iPSCs in large volume suspension cultures using bioreactors we can now cryopreserve iPSCs in large volumes. This allows us to significantly reduce the costs and time required to scale up iPSCs before differentiating to final cell product. Notably, we can create these suspension iPSCs with excellent cell quality and high genetic integrity. This invention demonstrates data to overcome those issues by efficient scaling up manufacturing of stem cell using bioreactors with standardized criteria and critical process parameters.

Described herein, we constructed a scalable human suspension iPSC sphere culture system by adjusting different feeding methods and seeding density using bench scale bioreactor. We developed reliable in-process controls to monitor the quality of the iPSCs by analyzing metabolite profiles dynamically over multiple passages and co-related to pluripotency marker expression to assess their quality. First, we adjusted cell adaption from 2D (adherent cultures) to 3D (suspension cultures) with different initial seeding cell density and identified how seeding density affects the suspension cell adaptation process. Next, to establish GMP media feeding regimen, we tested several feeding patterns and monitored the iPSC sphere size, expansion rate, pluripotency retention and identified critical metabolite parameters like ammonia and lactate to standardize iPSC maintenance and expansion. We also monitored cell viability and growth recovery after cryopreservation of suspension iPSCs. Taken together, our results using bench-scale bioreactor indicate that our suspension iPSC culture method can yield scalable quantity of GMP-compliant iPSCs with well-established in-process controls, and now ready to advance to manufacture quality suspension iPSCs at a large scale (e.g., 50 L Sartorius STR Bioreactor).

Various embodiments of the present invention are based, at least in part, on these findings.

Various embodiments provide for a method of adapting adherent induced pluripotent stem cells (iPSCs) to suspension iPSCs, comprising:
(i) seeding about 75,000-800,000 iPSCs per ml cGMP feeder-free iPSC maintenance medium;
(ii) culturing the iPSCs for about 2.5-3.5 days to allow the formation of iPSC spheres;
(iii) culturing the iPSC spheres for about 3.5-4.5 days, feeding the iPSC spheres about daily with the cGMP feeder-free iPSC maintenance medium;
(iv) passaging the iPSC spheres;
(v) culturing about 80,000-120,000 iPSC spheres for about 3.5-4.5 days in the cGMP feeder-free iPSC maintenance medium;
(vi) culturing the iPSC sphere for about 2.5-3.5 days, feeding the culture about daily with the cGMP feeder-free iPSC maintenance medium;
(vii) repeating steps (iv)-(vi) at least once.

In various embodiments, about 100,000 iPSCs per ml are seeded. In various embodiments, about 200,000 iPSCs per ml are seeded. In various embodiments, about 300,000 iPSCs per ml are seeded. In various embodiments, about 700,000 iPSCs per ml are seeded. In various embodiments, about 100,000-200,000 iPSCs per ml are seeded. In various embodiments, about 200,000-300,000 iPSCs per ml are seeded. In various embodiments, about 300,000-400,000 iPSCs per ml are seeded. In various embodiments, about 400,000-500,000 iPSCs per ml are seeded. In various embodiments, about 500,000-600,000 iPSCs per ml are seeded. In various embodiments, about 600,000-700,000 iPSCs per ml are seeded. In various embodiments, about 700,000-800,000 iPSCs per ml are seeded.

In various embodiments, passaging the iPSC spheres comprises:
collecting the iPSC spheres in a tube (e.g., a conical tube);
settling down iPSC spheres and removing extra medium;
adding about 4-6 ml dissociation solution, wherein the dissociation solution comprises cell detachment solution of proteolytic and collagenolytic enzymes and does not contain mammalian or bacterial derived products, and incubating in an about 36-37 C degree water bath for about 3-12 min;
dissociating iPSC spheres;
straining the iPSC spheres through an about 35-45 um cell strainer and collecting the strained iPSC spheres;
adding a quantity of cGMP feeder-free iPSC maintenance medium and a quantity of dissociation solution to the strained iPSC spheres;
spinning a conical tube comprising the strained iPSC spheres, cGMP feeder-free iPSC maintenance medium and dissociation solution at about 250-350×g for about 4-6 min;
removing supernatant and resuspending the iPSC spheres with fresh cGMP feeder-free iPSC maintenance medium.

In various embodiments, repeating steps (iv)-(vi) at least once comprises repeating steps (iv)-(vi) two to three times. In various embodiments, repeating steps (iv)-(vi) at least once comprises repeating steps (iv)-(vi) two to five times. In various embodiments, repeating steps (iv)-(vi) at least once comprises repeating steps (iv)-(vi) 6 to 10 times. In various embodiments, repeating steps (iv)-(vi) at least once comprises repeating steps (iv)-(vi) 11 to 15 times. In various embodiments, repeating steps (iv)-(vi) at least once comprises repeating steps (iv)-(vi) 16 to 20 times. In various embodiments, repeating steps (iv)-(vi) at least once comprises repeating steps (iv)-(vi) 21 to 30 times. In various embodiments, repeating steps (iv)-(vi) at least once comprises repeating steps (iv)-(vi) 31 to 40 times.

In various embodiments, the method comprises:
(i) seeding about 100,000-700,000 iPSCs per ml cGMP feeder-free iPSC maintenance medium;
(ii) culturing the iPSCs for about three days to allow the formation of iPSC spheres;
(iii) culturing the iPSC spheres for about four days, feeding the iPSC spheres daily with the cGMP feeder-free iPSC maintenance medium;
(iv) passaging the iPSC spheres;
(v) culturing about 100,000 iPSC spheres for about four days in the cGMP feeder-free iPSC maintenance medium;
(vi) culturing the iPSC sphere for about three days, feeding the culture daily with the cGMP feeder-free iPSC maintenance medium;
(vii) repeating steps (iv)-(vi) at least once.

In various embodiments, passaging the iPSC spheres comprises:
collecting the iPSC spheres in tube (e.g., a conical tube);
settling down iPSC spheres and removing extra medium;
adding about 5 ml dissociation solution, wherein the dissociation solution comprises cell detachment solution of proteolytic and collagenolytic enzymes and does not contain mammalian or bacterial derived products, and incubating in a 37 degree water bath for about 5-10 min;
dissociating iPSC spheres;
straining the iPSC spheres through an about 40 um cell strainer and collecting the strained iPSC spheres;
adding approximately equal amounts of cGMP feeder-free iPSC maintenance medium and dissociation solution to the strained iPSC spheres;
spinning a conical tube comprising the strained iPSC spheres, cGMP feeder-free iPSC maintenance medium and dissociation solution at about 300×g for about 5 min;
removing supernatant and resuspending the iPSC spheres with fresh cGMP feeder-free iPSC maintenance medium.

In various embodiments, repeating steps (iv)-(vi) at least once comprises repeating steps (iv)-(vi) two to three times. In various embodiments, repeating steps (iv)-(vi) at least once comprises repeating steps (iv)-(vi) two to five times. In various embodiments, repeating steps (iv)-(vi) at least once comprises repeating steps (iv)-(vi) 6 to 10 times. In various embodiments, repeating steps (iv)-(vi) at least once comprises repeating steps (iv)-(vi) 11 to 15 times. In various embodiments, repeating steps (iv)-(vi) at least once comprises repeating steps (iv)-(vi) 16 to 20 times. In various embodiments, repeating steps (iv)-(vi) at least once comprises repeating steps (iv)-(vi) 21 to 30 times. In various embodiments, repeating steps (iv)-(vi) at least once comprises repeating steps (iv)-(vi) 31 to 40 times.

In various embodiments, the method further comprises counting the iPSC spheres.

In various embodiments, the method further comprises banking the iPSC spheres or further passaging the iPSC spheres. In various embodiments, further passaging the iPSC spheres comprise further passaging the cells 1-5 times, 6-10 times, 11-15 times, 16-20 times, 21-25 times, 26-30 times, or 31-40 times.

In various embodiments, the suspension culture is in an about 30 ml bioreactor. In various embodiments, the suspension culture is in an about 15 ml to 2000 L bioreactor. In various embodiments, the suspension culture is in an about 50 L, 75 L, 100 L, 250 L, 500 L, 1000 L, 1500 L or 2000 L bioreactor. In various embodiments, the suspension culture is in an about 50 ml, 75 ml, 100 ml, 250 ml, 500 ml, 750 ml or 1000 ml bioreactor. In various embodiments, the suspension culture is in an about 1 L, 2.5 L, 5 L, 7.5 L, 10 L, or 25 L bioreactor.

The bioreactor can be a stirred tank bioreactor (e.g., stirred-impeller mixed bioreactor). In various embodiments, the speed of agitation is about 60-70 RPM. In various embodiments, the speed of agitation is about 50-55 RPM, 55-60 RPM, 60-65RPM, 65-70 RPM, 70-75 RPM, or 75-80 RPM. In various embodiments, the speed of agitation is about 40-90 RPM. In various embodiments, the speed of agitation is about 40-50 RPM, 50-60 RPM, 60-70 RPM or 80-90 RPM.

In various embodiments, the method results in an about 2.5-fold to about 18.9-fold expansion of the iPSCs after about three passages. In various embodiments, the method results in an about 2.5-fold to about 16.5-fold expansion of the iPSCs after about three passages. In various embodiments, the method results in an about 18.9-fold expansion of the iPSCs after about three passages.

In various embodiments, the method results in an about 2-fold, 5-fold, 10-fold, 15-fold, 20-fold, 25-fold or 30-fold expansion of the iPSCs after about three passages. In various embodiments, the method results in an at least 2-fold, 5-fold, 10-fold, 15-fold, 20-fold, 25-fold or 30-fold expansion of the iPSCs after about three passages.

In various embodiments, the method further comprises performing cytogenetic analysis on the expanded iPSCs. For example, G-banding karyotyping of the iPSCs can be performed. In various embodiments, the expanded iPSCs do not have numerical or structural abnormality. In various embodiments, the expanded iPSCs demonstrate no clinically significant abnormality.

In various embodiments, the culture conditions are monitored for glucose, lactate and ammonium concentrations. In various embodiments, the culture conditions are maintained with glucose, lactate and ammonium concentrations of up to 1 g/L, 2 g/L and 2.5 mmol/L, respectively.

Various embodiments of the invention provide for a method of culturing induced pluripotent stem cells (iPSCs) in suspension medium, comprising
(i) seeding about 80,000-120,000 iPSCs spheres per ml in cGMP feeder-free iPSC maintenance medium for about 3.5-4.5 days, wherein the iPSCs have been adapted from adherent iPSCs to suspended iPSCs;
(ii) culturing the iPSC spheres for about 2.5-3.5 days, feeding the iPSC sphere culture daily with cGMP feeder-free iPSC maintenance medium; and
(iii) passaging the iPSC spheres.

In various embodiments, the method further comprises repeating steps (i)-(iii) at least once. In various embodiments, repeating steps (i)-(iii) at least once comprises repeating steps (i)-(iii) two to five times. In various embodiments, repeating steps (i)-(iii) at least once comprises repeating steps (i)-(iii) 6 to 10 times. In various embodiments, repeating steps (i)-(iii) at least once comprises repeating steps (i)-(iii) 11 to 15 times. In various embodiments, repeating steps (i)-(iii) at least once comprises repeating steps (i)-(iii) 16 to 20 times. In various embodiments, repeating steps (i)-(iii) at least once comprises repeating steps (i)-(iii) 21 to 30 times. In various embodiments, repeating steps (i)-(iii) at least once comprises repeating steps (i)-(iii) 31 to 40 times.

In various embodiments, the method further comprises repeating steps (ii)-(iii) at least once. In various embodiments, repeating steps (ii)-(iii) at least once comprises repeating steps (ii)-(iii) two to five times. In various embodiments, repeating steps (ii)-(iii) at least once comprises repeating steps (ii)-(iii) 6 to 10 times. In various embodiments, repeating steps (ii)-(iii) at least once comprises repeating steps (ii)-(iii) 11 to 15 times. In various embodiments, repeating steps (ii)-(iii) at least once comprises repeating steps (ii)-(iii) 16 to 20 times. In various embodiments, repeating steps (ii)-(iii) at least once comprises repeating steps (ii)-(iii) 21 to 30 times. In various embodiments, repeating steps (ii)-(iii) at least once comprises repeating steps (ii)-(iii) 31 to 40 times.

In various embodiments, passaging the iPSC spheres comprises:
collecting the iPSC spheres in tube (e.g., a conical tube);
settling down spheres and removing extra medium;
adding about 4-6 ml dissociation solution and incubating in an about 36-38 C degree water bath for about 3-12 min;
dissociating iPSC spheres;
straining the iPSC spheres through an about 35-45 um cell strainer;
adding a quantity of cGMP feeder-free iPSC maintenance medium and a quantity of dissociation solution;
spinning the tub at about 250-350×g for about 4-6 min;
removing supernatant and resuspend the iPSC spheres with fresh cGMP feeder-free iPSC maintenance medium; and
counting the iPSC spheres.

In various embodiments, about 100,000, 300,000 or 700,000 iPSCs per ml are seeded. In various embodiments, about 100,000-300,000 iPSCs per ml are seeded. In various embodiments, about 300,000-500,000 iPSCs per ml are seeded. In various embodiments, about 500,000-700,000 iPSCs per ml are seeded.

In various embodiments, the method comprises
(i) seeding about 100,000 iPSCs spheres per ml in cGMP feeder-free iPSC maintenance medium for about four days, wherein the iPSCs have been adapted from adherent iPSCs to suspended iPSCs;
(ii) culturing the iPSC spheres for about three days, feeding the iPSC sphere culture daily with cGMP feeder-free iPSC maintenance medium; and
(iii) passaging the iPSC spheres.

In various embodiments, the method further comprises repeating steps (i)-(iii) at least once. In various embodiments, repeating steps (i)-(iii) at least once comprises repeating steps (i)-(iii) two to five times. In various embodiments, repeating steps (i)-(iii) at least once comprises repeating steps (i)-(iii) 6 to 10 times. In various embodiments, repeating steps (i)-(iii) at least once comprises repeating steps (i)-(iii) 11 to 15 times. In various embodiments, repeating steps (i)-(iii) at least once comprises repeating steps (i)-(iii) 16 to 20 times. In various embodiments, repeating steps (i)-(iii) at least once comprises repeating steps (i)-(iii) 21 to 30 times. In various embodiments, repeating steps (i)-(iii) at least once comprises repeating steps (i)-(iii) 31 to 40 times.

In various embodiments, the method further comprises repeating steps (ii)-(iii) at least once. In various embodiments, repeating steps (ii)-(iii) at least once comprises repeating steps (ii)-(iii) two to five times. In various embodiments, repeating steps (ii)-(iii) at least once comprises repeating steps (ii)-(iii) 6 to 10 times. In various embodiments, repeating steps (ii)-(iii) at least once comprises repeating steps (ii)-(iii) 11 to 15 times. In various embodiments, repeating steps (ii)-(iii) at least once comprises repeating steps (ii)-(iii) 16 to 20 times. In various embodiments, repeating steps (ii)-(iii) at least once comprises repeating steps (ii)-(iii) 21 to 30 times. In various embodiments, repeating steps (ii)-(iii) at least once comprises repeating steps (ii)-(iii) 31 to 40 times.

In various embodiments, passaging the iPSC spheres comprises:
collecting the iPSC spheres in tube (e.g., a conical tube);
settling down spheres and removing extra medium;
adding about 5 ml dissociation solution and incubating in an about 37 C degree water bath for about 5-10 min;
dissociating iPSC spheres;
straining the iPSC spheres through an about 40 um cell strainer;
adding approximately equal amounts of cGMP feeder-free iPSC maintenance medium and dissociation solution;
spinning the tub at about 300×g for about 5 min;
removing supernatant and resuspend the iPSC spheres with fresh cGMP feeder-free iPSC maintenance medium; and
counting the iPSC spheres.

In various embodiments, the method further comprises banking the cells or further passaging the cells. In various embodiments, further passaging the cells comprise further passaging the cells 1-5 times, 6-10 times, 11-15 times, 16-20 times, 21-25 times, 26-30 times, or 31-40 times.

In various embodiments, dissociating the cells comprises dissociating with force by pipetting.

In various embodiments, the suspension culture is in an about 30 ml bioreactor. In various embodiments, the suspension culture is in an about 15 ml to 2000 L bioreactor. In various embodiments, the suspension culture is in an about 50 L, 75 L, 100 L, 250 L, 500 L, 1000 L, 1500 L or 2000 L bioreactor. In various embodiments, the suspension culture is in an about 50 ml, 75 ml, 100 ml, 250 ml, 500 ml, 750 ml or 1000 ml bioreactor. In various embodiments, the suspension culture is in an about 1 L, 2.5 L, 5 L, 7.5 L, 10 L, or 25 L bioreactor.

The bioreactor can be a stirred tank bioreactor (e.g., stirred-impeller mixed bioreactor). In various embodiments, the speed of agitation is about 60-70 RPM. In various embodiments, the speed of agitation is about 50-55 RPM, 55-60 RPM, 60-65 RPM, 65-70 RPM, 70-75 RPM, or 75-80 RPM. In various embodiments, the speed of agitation is about 40-90 RPM. In various embodiments, the speed of agitation is about 40-50 RPM, 50-60 RPM, 60-70 RPM or 80-90 RPM.

In various embodiments, the method results in an about 2.5-fold to about 18.9-fold expansion of the iPSCs after about three passages. In various embodiments, the method results in an about 2.5-fold to about 16.5-fold expansion of the iPSCs after about three passages. In various embodiments, the method results in an about 18.9-fold expansion of the iPSCs after about three passages.

In various embodiments, the method results in an about 2-fold, 5-fold, 10-fold, 15-fold, 20-fold, 25-fold or 30-fold expansion of the iPSCs after about three passages. In various embodiments, the method results in an at least 2-fold, 5-fold, 10-fold, 15-fold, 20-fold, 25-fold or 30-fold expansion of the iPSCs after about three passages.

In various embodiments, the method further comprises performing cytogenetic analysis on the expanded iPSCs. For example, G-banding karyotyping of the iPSCs can be performed. In various embodiments, the expanded iPSCs do not have numerical or structural abnormality. In various embodiments, the expanded iPSCs demonstrate no clinically significant abnormality.

In various embodiments, the culture conditions are monitored for glucose, lactate and ammonium concentrations. In various embodiments, the culture conditions are maintained with glucose, lactate and ammonium concentrations of up to 1 g/L, 2 g/L and 2.5 mmol/L, respectively.

Various embodiments provide for a method of cell preservation, comprising: dissociating iPSC spheres as single cells by using a dissociation solution and cryopreserving iPSC spheres with 3e7 cells/vial using a freezing reagent. In various embodiments, the method further comprises monitoring cell recovery by re-thawing the cryopreserved iPSC with 100 k/ml density in spinner. In various embodiments, the method further comprises using flow analysis to detect pluripotency after thawing. In various embodiments, flow analysis is performed about 2 weeks after thawing.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

To install scaling up system using bioreactor, used 3 different approaches 1) adherent culture to suspension culture adaption, 2) optimized feeding method with passing, 3) cell preservation using small scale bioreactor (30 ml).

For cell adaptation, firstly, we use 2 different hiPSC lines, 0003iCTR and 628iCTR, to identify optimal initial seeding density of this transitional cell culture system and monitor sphere morphology, size growth, cell expansion rate, pluripotency and media metabolic condition, providing the best seeding density for different iPSC lines and culture method to retain cellular properties. Next, the volume of media, culture interval and passing frequency are major considerable culture conditions for suspension feeding method. In order to establish the best feeding system for suspension culture, we tested several different feeding methods and validate final cells by our analytic approaches such as flow analysis, monitoring system of sphere growth, expansion rate, morphology, and confirm this validation with at least 3 technical and biological replicates. For NOVA metabolic media condition, additionally, we utilized the culture condition of adherent cell to standardize all parameters for suspension culture. Lastly, cell preservation method is important to overcome time and distance limit as well as globalization. We verified cell viability and proliferation recovery of post-thawed cells, indicating optimal cell freezing and thawing method for final cell products.

Results show that the seeding density and monitoring parameters for cell adaptation process. Pluripotency, cell expansion rate, morphology, size and metabolic media conditions are critical parameters to establish best feeding method for suspension culture. NOVA analysis indicates metabolic media condition during culture. Adherent culture (2D) provides substantial criteria for culture standard of suspension culture. Cell viability and recovery rate are important for cell product preservation.

### Example 2

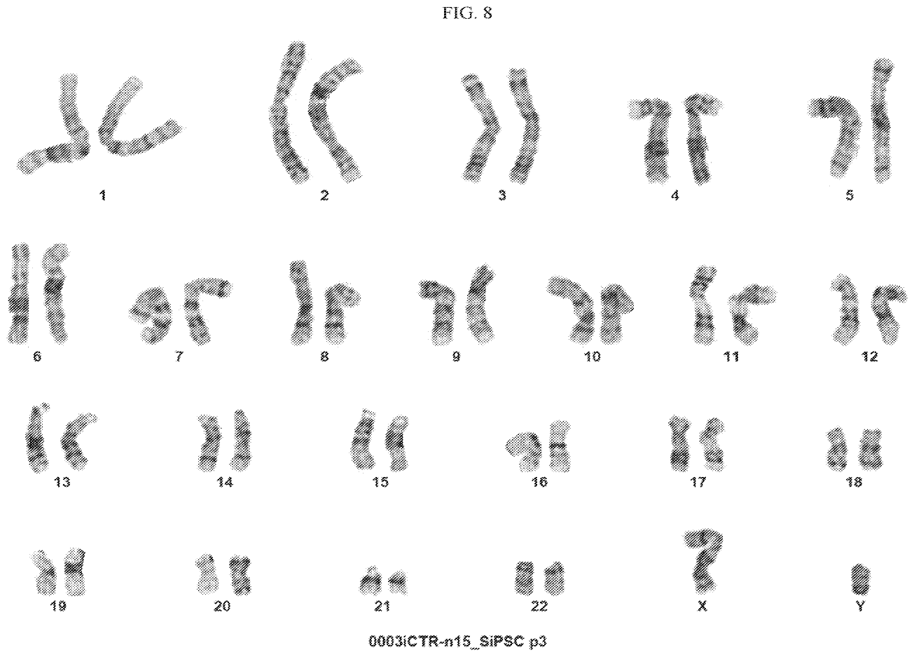

Cytogenetic analysis using G-banding at the 400 band level of resolution on slides of these cultured cells revealed a 46,XY normal male karyotype. No consistent numerical or structural abnormality was observed, and there was no demonstrable clinically significant abnormality. See FIG. 8.

### Example 3

### GMP-Grade SOP Describing Bioreactor Parameters for Efficient iPSC Scale-Up

Small-scale bioreactor systems have been set up to evaluate suspension iPSC culture parameters.

First set of experiments were performed using small-scale bioreactor to measure the cell expansion rate, sphere size, overall viability of iPSCs and culture media condition for optimized feeding method in bioreactor systems.

Suspension cultures have been performed to build a suspension-based cell bank for further process development. To achieve this goal, one of cell lines, 2AE8iCTR (healthy hiPSC), has been grown in suspension using small-scale bioreactor at different passage number and feeding methods to evaluate some initial process parameters.

The cell has been grown in 20 mL spinner flasks that are agitating at 70 RPM inside an incubator at 37 degrees Celsius with 5% CO₂. Initial seeding density had been established at 1e5 cells/mL while using mTeSR Plus with rock inhibitor supplementation. Our first goal was to identify the best feeding methods, providing maximal hiPSC expansion and pluripotency cells in efficient way. Therefore, we took advantage of diverse strategies to access 1) measuring sphere size, 2) cell growth expansion rate, 3) viability, 4) metabolite date from NOVA Flex2 and 5) Flow analysis by OCT4 and SSEA4 antibodies presenting pluripotency.

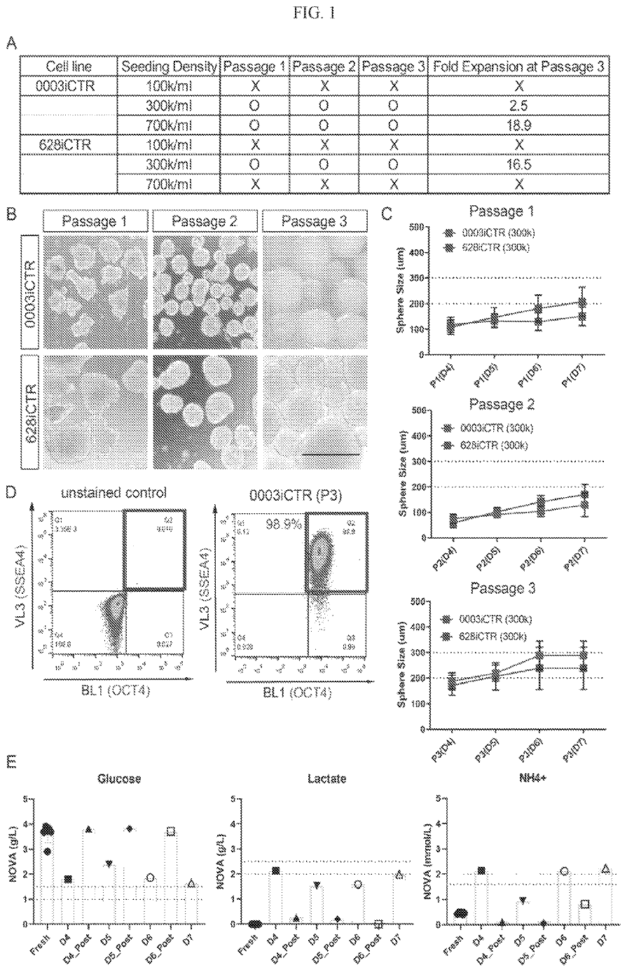

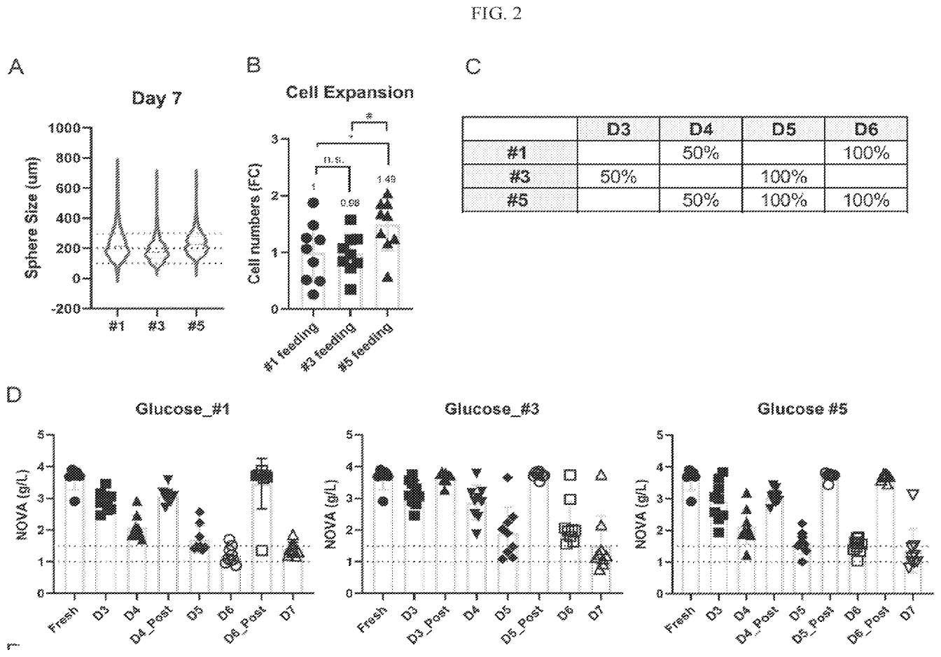

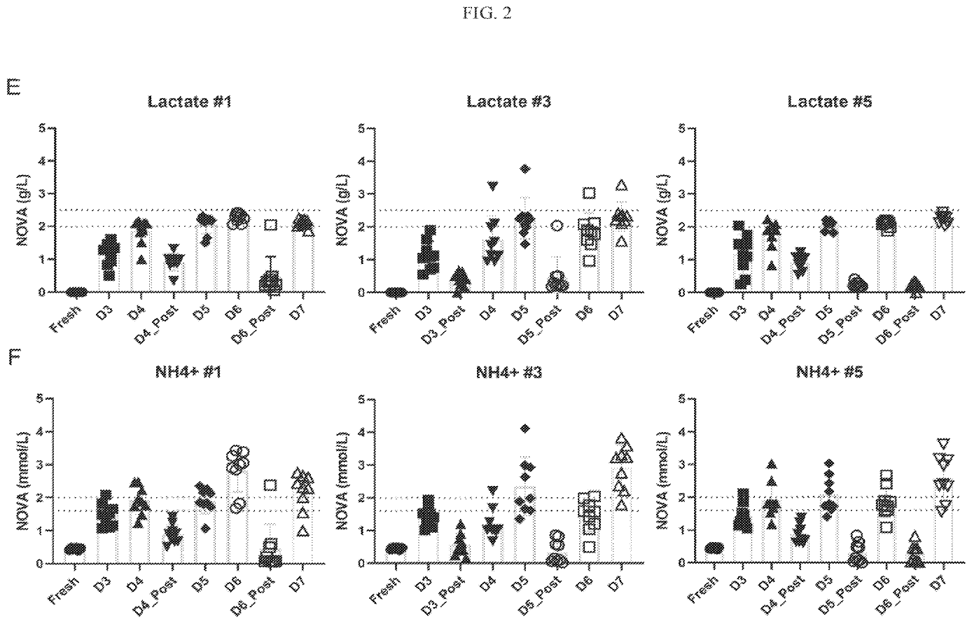

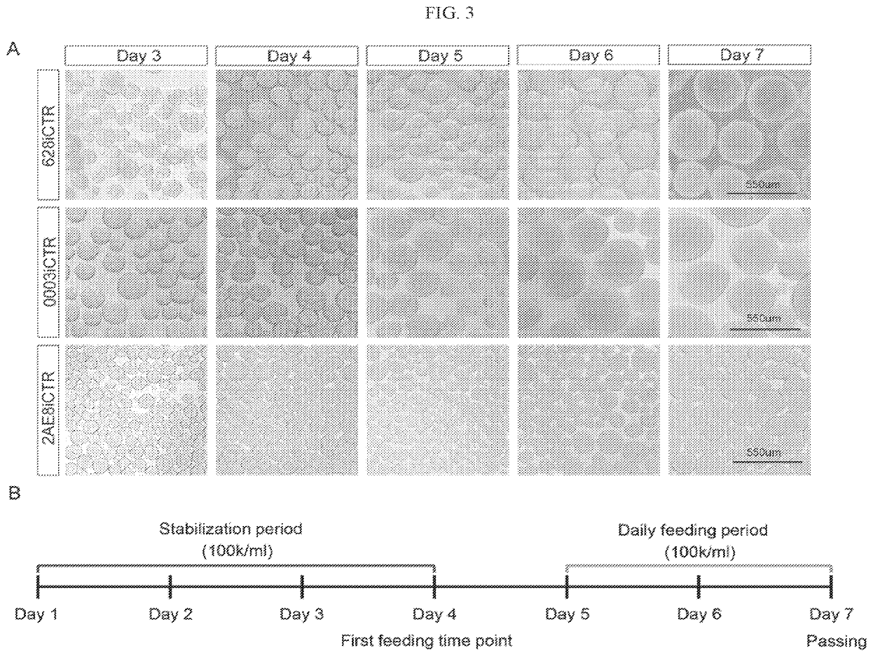

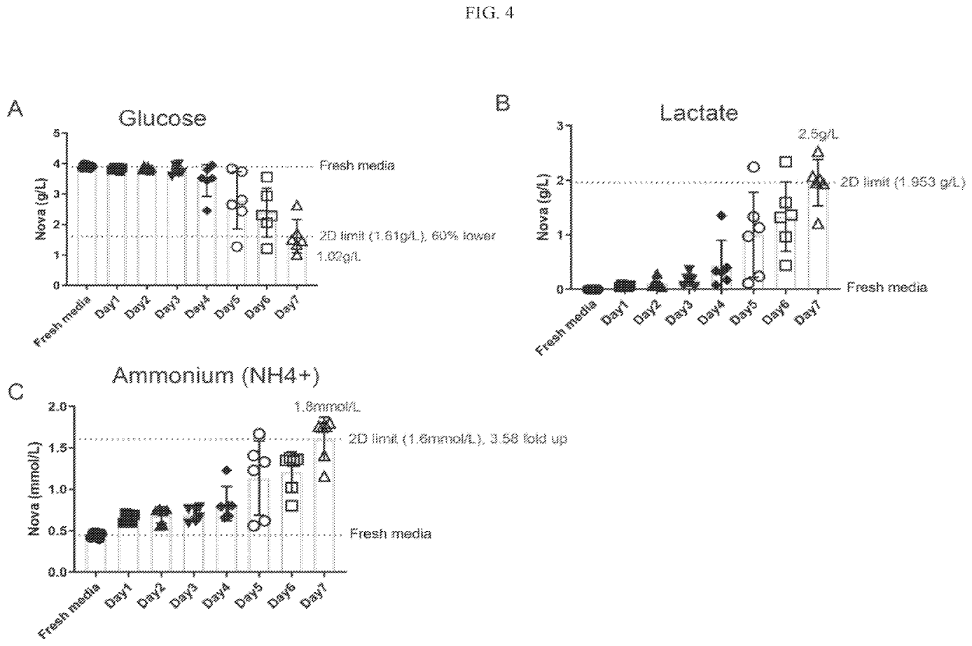

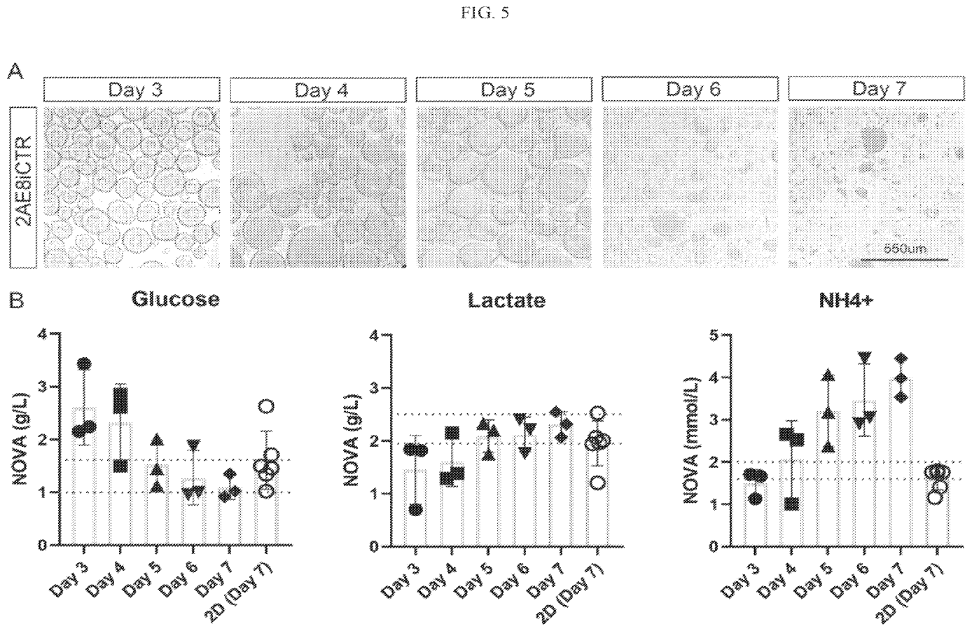

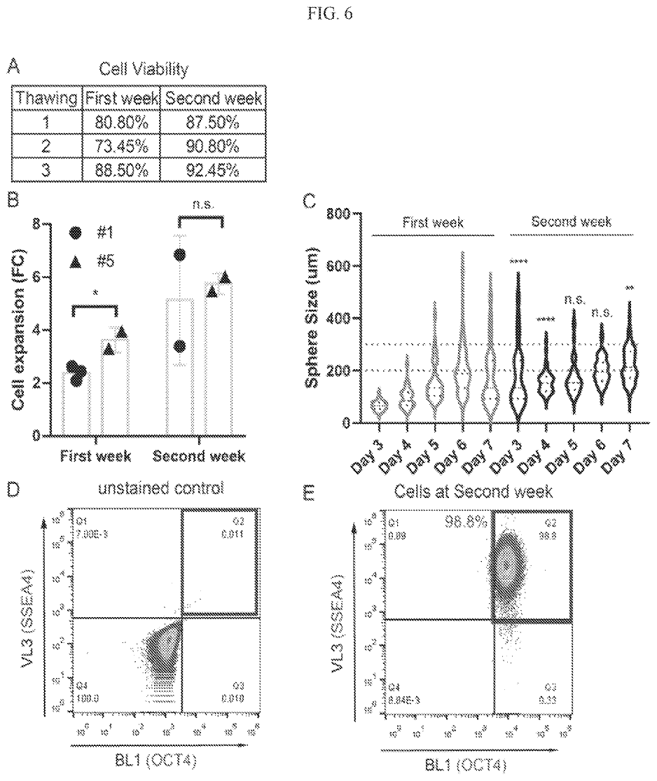

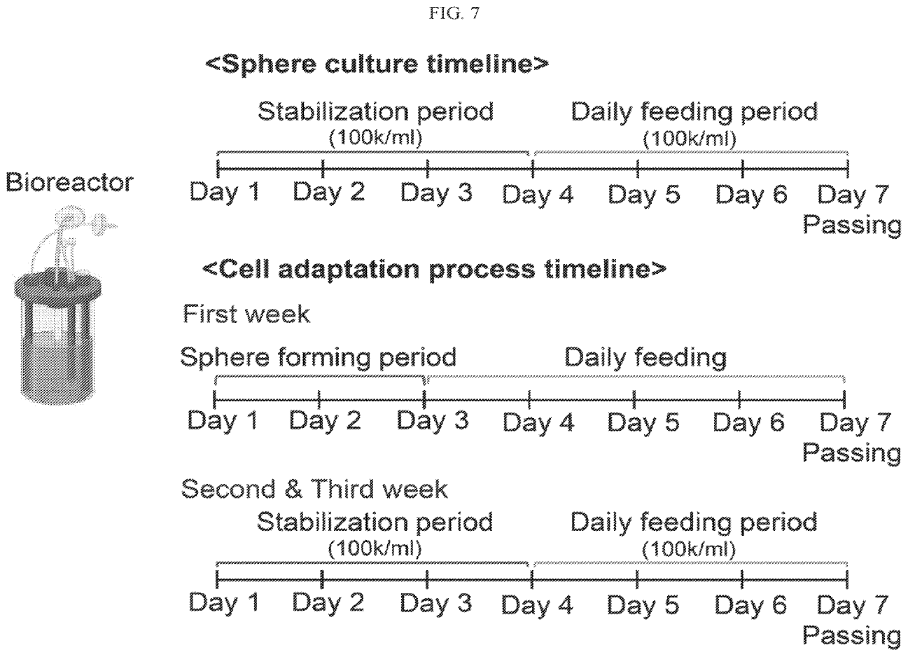

To establish optimal feeding method for SiPSC culture, we selected 7 different feeding methods for 7 days (#1: standard method, #7: negative control) with at 1e5 cells/mL seeding density using one line (2AE8iCTR) depending on amount and frequency of mTeSR Plus media (see FIG. 1A). Feeding started at post-3 days of cell seeding and harvested all spheres from each condition at day 7. Fold expansion result shows that frequent feeding methods culture day 4-6 might be critical for robust hiPSC proliferation.

During culture time, we also measured sphere sizes that indicates growth rate of each feeding condition. All conditions show gradual growth of spheres by different time point with ideal morphology. Especially, sphere size of #1-6 feeding conditions is within 200-300 um and the size is never exceed 400 um, avoiding hypoxia by improper metabolic penetration.

Although we closely access to optimal SiPSC culture by feeding condition, retaining pluripotency is most important criteria. Thus, we attempted to establish flow cytometry condition to identify pluripotent marker expression using OCT4 and SSEA4 antibodies. Our cultured SiPSC shows highly co-expressed two maker expression (96.8%) after dissociation as much as hESC (99%, set as positive control), presenting successful set up of flow method/condition and optimal SiPSC culture system.

Next, media condition is also critical to optimize the best feeding condition and this condition was able to access measuring by metabolic values using NOVA Flex2. First of all, we were required to set up standard parameter. Thus, we plated hiPSC (6 different lines or clones) in the plate coating with human laminin 521 (LN521) substrate and measured metabolic condition in the media at day 1-7. In fact, using NOVA Flex2, we can measure Sodium, Potassium, Calcium, Osmolality, pH, pCO2, pO2, Glucose, Glutamine, Glutamate, Lactate, and Ammonium, but we selected 3 major factors (Limit value of Glucose: 1 g/L, Lactate: 2 g/L and Ammonium: 2.5 mmol/L). Based on those actionable parameters from 2D culture condition/literatures, we measured metabolic condition in the media of SiPSC. #5 feeding condition shows highest (1.8 fold change upon cell expansion of #1) cell expansion among 7 different feed methods. We speculated that cells are explosively expanded during day 4-7, Occasionally, metabolic values over the limit in #1 and 5, but this is temporal event (#7 is negative control without feeding schedule). Overall, our current progress is closely reached to achieve

To determine bioreactor parameters for efficient iPSC scale up, we established actionable parameter based on 2D culture/literature, pluripotency profiling by flow analysis, and several feeding condition showing optimal sphere growth rate and morphology.

### Example 4

### Validate cGMP Compliant Harvesting and Preservation Methods for iPSCs

For cell banking method, cell viability, recovery rate of pluripotency (flow analysis), proliferate (overall cell growth) and differentiation potent are considered as major parameters.

As first set of experiment, we monitored cell growth rate (sphere size and cell expansion) and pluripotency by flow analysis post-thawing.

In order to repeat and confirm, our previous results that aim to establish best feeding methods is required to test using different iPSC lines. As previous cell culture condition, the cell has been grown in 20 mL spinner flasks that are agitating at 70 RPM inside an incubator at 37 degrees Celsius with 5% CO2. Initial seeding density had been established at 1e5 cells/mL while using mTeSR Plus with rock inhibitor supplementation. Using previous same culture condition with selected feeding methods, we tested same parameters (sphere morphology, size growth, expansion, pluripotency and media metabolomic analysis) in different iPSC line (0003iCTR), providing identical results as shown in previous. For cell adaptation process (Subtask 1.3.2), we tested 3 different initial seeding density of 2 different iPSC (0003iCTR and 628iCTR) for first week and verified cell condition and growth by parameters as we established. This preliminary data shows the idea about critical parameters in the process and time period to complete the adaption. For cell banking, we cryopreserved cells with 3e7 cells/vial using CS10 (Cryostor, StemCell), and monitored cell viability, cell growth by weekly (expansion and sphere size) and pluripotency, showing cells are successfully recovered by time in our current method. It provides the initial idea about cell numbers for preservation and time for complete recovery for further test.

### Example 5

### Validate cGMP Compliant Harvesting and Preservation Methods for iPSCs

In order to repeat and confirm, our previous results (2AE8iCTR) that aim to establish best feeding methods is required to test using different iPSC lines. Addition to this, our negative control (non-feeding) provides critical metabolite for media condition monitoring by NOVA analysis. As previous cell culture condition, the cell has been grown in 20 mL spinner flasks that are agitating at 70 RPM inside an incubator at 37 degrees Celsius with 5% CO₂. Initial seeding density had been established at 1e5 cells/mL while using mTeSR Plus with rock inhibitor supplementation. Using previous same culture condition with selected feeding methods, we tested same parameters (sphere morphology, size growth, expansion, pluripotency and media metabolomic analysis) in different iPSC line (0003iCTR and 628iCTR), providing identical results as shown in previous. For cell adaptation process, we tested 3 different initial seeding density of 2 different iPSC (0003iCTR and 628iCTR) for first week and verified cell condition and growth by parameters as we established. This preliminary data shows the idea about critical parameters in the process and time period to complete the adaption. For cell banking, we cryopreserved cells with 3e7 cells/vial using CS10 (Cryostor, StemCell), and monitored cell viability, cell growth by weekly (expansion and sphere size) and pluripotency, showing cells are successfully recovered by time in our current method. It provides the initial idea about cell numbers for preservation and time for complete recovery for further test.

### Example 6

Cultured sphere cells were dissociated as single cells by accutase, and then cryopreserved iPSC with 3e7 cells/vial using CS10 (Cryostor, StemCell). To monitor cell recovery, we re-thawed those cryopreserved iPSC with 100 k/ml density in spinner. Cell viability shows average 80.92% in first week (thawing) and 90.25 in second week (one week after thawing). This recovery aspect is correlated with data of cell expansion and sphere size. Finally, flow analysis also presents cells still retain the high level of pluripotency after 2 weeks thawing. Those results provide that the basic idea in terms of cell number for cryopreservation and expected viability, time to complete recovery. Further study will verify our future plans to demonstrate our banking system.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of adapting adherent induced pluripotent stem cells (iPSCs) to suspension iPSCs, comprising:
(i) seeding about 75,000-800,000 iPSCs per ml cGMP feeder-free iPSC maintenance medium;
(ii) culturing the iPSCs for about 2.5-3.5 days to allow the formation of iPSC spheres;
(iii) culturing the iPSC spheres for about 3.5-4.5 days, feeding the iPSC spheres about daily with the cGMP feeder-free iPSC maintenance medium;
(iv) passaging the iPSC spheres;
(v) culturing about 80,000-120,000 iPSC spheres for about 3.5-4.5 days in the cGMP feeder-free iPSC maintenance medium;
(vi) culturing the iPSC sphere for about 2.5-3.5 days, feeding the culture about daily with the cGMP feeder-free iPSC maintenance medium; and
(vii) repeating steps (iv)-(vi) at least once.

2. The method of claim 1, wherein passaging the iPSC
spheres comprises: collecting the iPSC spheres in a tube;
settling down iPSC spheres and removing extra medium;
adding about 4-6 ml dissociation solution, wherein the dissociation solution comprises cell detachment solution of proteolytic and collagenolytic enzymes and does not contain mammalian or bacterial derived products, and incubating in an about 36-37 C degree water bath for about 3-12 min;
dissociating iPSC spheres;
straining the iPSC spheres through an about 35-45 um cell strainer and collecting the strained iPSC spheres;
adding a quantity of cGMP feeder-free iPSC maintenance medium and a quantity of dissociation solution to the strained iPSC spheres;
spinning a conical tube comprising the strained iPSC spheres, cGMP feeder-free iPSC maintenance medium and dissociation solution at about 250-350×g for about 4-6 min; and
removing supernatant and resuspending the iPSC spheres with fresh cGMP feeder-free iPSC maintenance medium.

3. The method of claim 1, comprising:
(i) seeding about 100,000-700,000 iPSCs per ml cGMP feeder-free iPSC maintenance medium;
(ii) culturing the iPSCs for about three days to allow the formation of iPSC spheres;
(iii) culturing the iPSC spheres for about four days, feeding the iPSC spheres daily with the cGMP feeder-free iPSC maintenance medium;
(iv) passaging the iPSC spheres;
(v) culturing about 100,000 iPSC spheres for about four days in the cGMP feeder-free iPSC maintenance medium;
(vi) culturing the iPSC sphere for about three days, feeding the culture daily with the cGMP feeder-free iPSC maintenance medium; and
(vii) repeating steps (iv)-(vi) at least once.

4. The method of claim 3, wherein passaging the iPSC
spheres comprises: collecting the iPSC spheres in a tube;
settling down iPSC spheres and removing extra medium;
adding about 5 ml dissociation solution, wherein the dissociation solution comprises cell detachment solution of proteolytic and collagenolytic enzymes and does not contain mammalian or bacterial derived products, and incubating in a 37 degree water bath for about 5-10 min;
dissociating iPSC spheres;
straining the iPSC spheres through an about 40 um cell strainer and collecting the strained iPSC spheres;
adding approximately equal amounts of cGMP feeder-free iPSC maintenance medium and dissociation solution to the strained iPSC spheres;
spinning a conical tube comprising the strained iPSC spheres, cGMP feeder-free iPSC maintenance medium and dissociation solution at about 300×g for about 5 min; and removing supernatant and resuspending the iPSC spheres with fresh cGMP feeder-free iPSC maintenance medium.

5. The method of claim 1, further comprising counting the iPSC spheres.

6. The method of claim 5, further comprising banking the iPSC spheres or further passaging the iPSC spheres.

7. The method of claim 1, wherein the suspension culture is in an about 30 ml bioreactor.

8. A method of culturing induced pluripotent stem cells (iPSCs) in suspension medium, comprising
(i) seeding about 80,000-120,000 iPSCs spheres per ml in cGMP feeder-free iPSC maintenance medium for about 3.5-4.5 days, wherein the iPSCs have been adapted from adherent iPSCs to suspended iPSCs;
(ii) culturing the iPSC spheres for about 2.5-3.5 days, feeding the iPSC sphere culture daily with cGMP feeder-free iPSC maintenance medium; and
(iii) passaging the iPSC spheres.

9. The method of claim 8, wherein passaging the iPSC
spheres comprises: collecting the iPSC spheres in a tube;
settling down spheres and removing extra medium;
adding about 4-6 ml dissociation solution and incubating in an about 36-38 C degree water bath for about 3-12 min;
dissociating iPSC spheres;
straining the iPSC spheres through an about 35-45 um cell strainer;
adding a quantity of cGMP feeder-free iPSC maintenance medium and a quantity of dissociation solution;
spinning the tub at about 250-350×g for about 4-6 min;
removing supernatant and resuspend the iPSC spheres with fresh cGMP feeder-free iPSC maintenance medium; and
counting the iPSC spheres.

10. The method of claim 8, comprising
(i) seeding about 100,000 iPSCs spheres per ml in cGMP feeder-free iPSC maintenance medium for about four days, wherein the iPSCs have been adapted from adherent iPSCs to suspended iPSCs;
(ii) culturing the iPSC spheres for about three days, feeding the iPSC sphere culture daily with cGMP feeder-free iPSC maintenance medium; and
(iii) passaging the iPSC spheres.

11. The method of claim 8, wherein
passaging the iPSC spheres comprises: collecting the iPSC spheres in a tube;
settling down spheres and removing extra medium;
adding about 5 ml dissociation solution and incubating in a 37 C degree water bath for about 5-10 min;
dissociating iPSC spheres;
straining the iPSC spheres through an about 40 um cell strainer;
adding approximately equal amounts of cGMP feeder-free iPSC maintenance medium and dissociation solution;
spinning the tub at about 300×g for about 5 min;
removing supernatant and resuspend the iPSC spheres with fresh cGMP feeder-free iPSC maintenance medium; and
counting the iPSC spheres.

12. The method of claim 8, further comprising repeating steps (i)-(iii) at least once.

13. The method of claim 8, further comprising repeating steps (ii)-(iii) at least once.

14. The method of claim 8, further comprising banking the cells or further passaging the cells.

15. The method of claim 11, wherein dissociating the cells comprises dissociating with force by pipetting.

16. The method of claim 8, wherein the suspension culture is in an about 30 ml bioreactor.

17. The method of claim 8, wherein the suspension culture is in an about 30 ml bioreactor.
